(19) United States
(12) Patent Application Publication
(10) Pub. No.: US 2026/0157938 A1
(43) Pub. Date: Jun. 11, 2026

(54) Curable Composition for Use in a Process of Treating a Dental Situation in the Mouth of a Patient

(71) Applicant: Solventum Intellectual Properties Company, Maplewood, MN (US)

(72) Inventors: Gioacchino Raia, Tuerkenfeld (DE); Reinhold Hecht, Schondorf am Ammersee (DE)

(21) Appl. No.: 19/126,954

(22) PCT Filed: Nov. 6, 2023
(86) PCT No.: PCT/IB2023/061194

§ 371 Date: May 2, 2025

(30) Foreign Application Priority Data
Nov. 25, 2022 (EP) 22209563.0

(51) Int. Cl. A61K 6/62; A61C 5/77; A61K 6/15; A61K 6/887; B33Y 10/00; B33Y 40/20; B33Y 70/10; B33Y 80/00

(52) U.S. Cl. CPC A61K 6/62; A61C 5/77; A61K 6/15; A61K 6/887; B33Y 10/00; B33Y 40/20; B33Y 70/10; B33Y 80/00

(57) ABSTRACT

The invention relates to a curable composition for use in a process of treating a dental situation in the mouth of a patient, the curable composition comprising radiation-curable component(s), photo-initiator(s), the photo-initiator(s) exhibiting an absorption in the UV light region, and an absorption in the visible light region, the absorption in the UV light region being stronger than the absorption in the visible light region, optionally filler(s), optionally additive(s), the process comprising the steps of additive manufacturing a dental or orthodontic article layer-by-layer using radiation with a wavelength in the UV light region, attaching the dental or orthodontic article to a surface of hard dental tissue or a dental material, applying radiation with a wavelength in the visible light region to the dental or orthodontic article.

### FIELD

The invention relates to a curable composition for use in a process of treating a dental situation in the mouth of a patient and a related kit. The curable composition comprises radiation-curable component(s), a photo-initiator with a specific absorption spectrum, optionally filler(s) and additive(s) and is curable by UV light and visible light.

### BACKGROUND

Using additive-manufacturing techniques, in particular 3D-printing for producing dental articles is known in the art. However, in particular articles produced by 3D-printing cannot be directly used but typically require post-processing steps.

Post-processing typically comprises steps like cleaning or removing uncured resin from the article removed from the printing bath and removing pins for supporting the article during the printing process.

Further, the 3D-printed articles typically need to be post-cured to obtain an article with sufficient mechanical properties. Depending on the use, the conditions used for the 3D-printing process and the post-curing step are different, in particular as regards the wavelength of the radiation used for curing.

In the dental field, the curing of radiation-curable compositions in the mouth of a patient may not be done with UV light, even if this would be more effective, whereas the 3D-printing is typically done with UV light.

Thus, depending on the intended use, adequate photo-initiators need to be selected which are sensitive in the respective wavelengths of the light used for either 3D-printing and post-curing.

For addressing this need, radiation-curable compositions have been suggested containing two different kinds of photo-initiators.

E.g., WO 2013/153183 A2 (Ivoclar) describes the use of a composite resin composition containing: (a) at least one poly-reactive binder; (b) a first photo-polymerization initiator with an absorption maximum at a wavelength of less than 400 nm; (c) a second photo-polymerization initiator with an absorption maximum at a wavelength of at least 400 nm; and (d) an absorber with an absorption maximum at a wavelength of less than 400 nm for the stereo-lithographic production of a dental formed component on the basis of composite resin.

### DESCRIPTION OF INVENTION

However, there is still a need for a radiation-curable composition which on the one hand can be economically processed by an additive-manufacturing process to obtain an article having adequate mechanical properties and on the other hand allows the provision of an article which still contains sufficiently high amounts of uncured moieties being available for a light-induced adhesively fixing of the article to hard dental tissue.

If desired, the mechanical properties of the article obtained by the additive-manufacturing process should also enable a mechanical manipulation of the 3D-printed article before further processing, such as trimming or cutting.

Ideally, also the extent of undesired light scattering during the additive-manufacturing process should be reduced.

Further, it can be desirable if the 3D-printed article has a high flexibility after the 3D-printing step which would allow an easier placing of the 3D-printed article on another subject, a subject having undercuts.

At least one of the above-mentioned objectives is addressed by the invention described in the present text and claims.

In particular, the invention relates to a curable composition for use in a process of treating a dental situation in the mouth of a patient,
the curable composition comprising
   radiation-curable component(s),
   photo-initiator(s), the photo-initiator(s) exhibiting an absorption in the UV light region, and
   an absorption in the visible light region, the absorption in the UV light region being stronger
   than the absorption in the visible light region,
   optionally filler(s),
   optionally additive(s),
the process comprising the steps of
   additive-manufacturing a dental or orthodontic article layer-by-layer using radiation with a
   wavelength in the UV light region,
   attaching the dental or orthodontic article to a surface of hard dental tissue or a dental material,
   applying radiation with a wavelength in the visible light region to the dental or orthodontic article.

The invention also relates to a kit of parts comprising the curable composition for use described in the present text, a dental adhesive or a dental cement, optionally a dental positioning tray, and optionally an instruction of use.

Moreover, the invention relates to a pre-cured composition obtainable by processing the curable composition described in the present text in an additive-manufacturing process, the pre-cured composition having the shape of a dental or orthodontic article.

### FIGURES

FIG. 1 shows the UV/VIS spectrum of camphor quinone.

FIG. 2 shows the UV/VIS spectrum ofphenyl-1,2-propanedione.

FIG. 3 shows the flexural strength testing of a 3D-printed article obtained from the curable composition described in the present text.

### DETAILED DESCRIPTION

The term “compound” or “component” is a chemical substance which has a certain molecular identity or is made of a mixture of such substances, e.g., polymeric substances.

A “hardenable or curable or polymerizable component” is any component which can be cured or solidified in the presence of a photo-initiator by radiation-induced polymerization. A hardenable component may contain only one, two, three or more polymerizable groups. Typical examples of polymerizable groups include unsaturated carbon groups, such as a vinyl group being present i.a. in a (methyl)acrylate group.

As used herein, “(meth)acryl” is a shorthand term referring to “acryl” and/or “methacryl”. For example, a “(meth) acryloxy” group is a shorthand term referring to either an acryloxy group (i.e., CH₂═CH—C(O)—O—) and/or a methacryloxy group (i.e., CH₂═C(CH₃)—C(O)—O—).

A “urethane group” is a group having the structure “—NH—CO—O—”.

As used herein, “hardening” or “curing” a composition are used interchangeably and refer to polymerization and/or crosslinking reactions including, for example, photo-polymerization reactions and chemical-polymerization techniques (e. g., chemical reactions forming radicals effective to polymerize ethylenically unsaturated compounds) involving one or more materials included in the composition.

“Radiation curable” shall mean that the component (or composition, as the case may be) can be cured by applying radiation, preferably electromagnetic radiation with a wavelength in the light spectrum range of 350 to 500 nm under ambient conditions and within a reasonable time frame (e.g., within about 15, 10 or 5 min).

“Dental article” means an article which is to be used in the dental or orthodontic field. A dental article has typically two different surface portions, an outer surface and an inner surface. The outer surface is the surface which is typically not in permanent contact with the surface of a tooth. In contrast thereto, the inner surface is the surface which is used for attaching or fixing the dental article to a tooth. If the dental article has the shape of a dental crown, the inner surface has typically a concave shape, whereas the outer surface has typically a convex shape. A dental article should not contain components which are detrimental to the patient's health and thus free of hazardous and toxic components being able to migrate out of the dental or orthodontic article.

“Orthodontic article” includes orthodontic brackets, buccal tubes, lingual retainers, orthodontic bands, bite openers, buttons, attachments and cleats.

“Hard dental tissue” includes enamel and dentin.

A “particle” means a substance being a solid having a shape which can be geometrically determined. The shape can be regular or irregular. Particles can typically be analysed with respect to e.g. particle size and particle size distribution.

“Agglomerated” is descriptive of a weak association of particles usually held together by charge or polarity and can be broken down into smaller entities. The specific surface of agglomerated particles does not essentially deviate from the specific surface of the primary particles the agglomerate is made of (cf. DIN 53206; 1972).

Agglomerated fillers are commercially available e.g., from Degussa, Cabot Corp or Wacker under the product designation Aerosil™, CAB—O-SIL™ and HDK.

A “non-agglomerated or discrete filler particles” means that the filler particles are present in the resin in a discrete, un-associated (i.e. non-agglomerated and non-aggregated) stage. If desired this can be proven by TEM microscopy.

Non-agglomerated nano-sized silicas are commercially available e.g., from Nalco Chemical Co. (Naperville, Ill.) under the product designation NALCO COLLOIDAL SILICAS e.g. NALCO products #1040, 1042, 1050, 1060, 2327 and 2329.

Non-agglomerated fillers are used and described e.g., in U.S. Pat. No. 8,329,776 B2 (Hecht et al.) The content of this reference is herewith incorporated by reference.

“Aggregated,” as used herein, is descriptive of a strong association of particles often bound together by, for example, residual chemicals treatment or partially sintering. The specific surface of aggregated particles is typically smaller than the specific surface of the primary particles the aggregate is made of (cf. DIN 53206; 1972).

A “nano-filler” is a filler, the individual particles thereof have a size in the region of nanometers, e.g., an average particle diameter of less than 200 nm or less than 100 nm or less than 50 nm. Useful examples are given in U.S. Pat. No. 6,899,948 (Zhang et al.) and U.S. Pat. No. 6,572,693 (Wu et al.) The content with regard to nano-sized silica particles is herein incorporated by reference.

“Additive-manufacturing” or “3D-printing” means processes comprising a layer-wise creation of an object from digital data. The articles can be of almost any shape or geometry and are produced from a 3-dimensional model or other electronic data source.

Many 3D-printing technologies exist, one of them being vat polymerization which uses a radiation curing step to make 3-dimensional articles. Examples of vat polymerization techniques include stereolithography (SLA) and digital light processing (DLP).

“Stereolithography” is an example of an additive-manufacturing technique where typically two motors are used for aiming a laser beam across the print area thereby solidifying the printing resin. This process breaks down the design, layer by layer, into a series of points.

“Digital light processing” is another example of an additive-manufacturing technique and typically comprises the use of a digital projector screen to flash an image of each layer across the building platform of the additive-manufacturing unit. The image is typically composed of square pixels, resulting in a layer formed from small rectangular bricks called voxels.

“UV light region” means light having a wavelength in the range of 350 to 410 nm.

“Visible light region” means light having a wavelength in the range of 440 to 500 nm.

“Ambient conditions” mean the conditions which the composition described in the present text is usually subjected to during storage and handling. Ambient conditions may, for example, be a pressure of 900 to 1,100 mbar, a temperature of 10 to 40° C. and a relative humidity of 10 to 100%. In the laboratory ambient conditions are typically adjusted to 20 to 25° C. and 1,000 to 1,025 mbar (at maritime level).

As used herein, “a”, “an”, “the”, “at least one” and “one or more” are used interchangeably. Also herein, the recitations of numerical ranges by endpoints include all numbers subsumed within that range (e.g., 1 to 5 includes 1, 1.5, 2, 2.75, 3, 3.80, 4, 5, etc.).

Adding an “(s)” to a term means that the term should include the singular and plural form. E.g., the term “additive(s)” means one additive and more additives (e.g., 2, 3, 4, etc.).

Unless otherwise indicated, all numbers expressing quantities of ingredients, measurement of physical properties such as described below and so forth used in the specification and claims are to be understood as being modified in all instances by the term “about”.

The terms “comprise” or “contain” and variations thereof do not have a limiting meaning where these terms appear in the description and claims. “Consisting essentially of” means that specific further components can be present, namely those which do not materially affect the essential characteristic of the article or composition. “Consisting of” means that no further components should be present. The term “comprise” shall include also the terms “consist essentially of” and “consists of”.

A composition is “essentially or substantially free of” a certain component, if the composition does not contain said component as an essential feature. Thus, said component is not wilfully added to the composition either as such or in combination with other components or ingredient of other components. A composition being essentially free of a certain component usually does not contain that component at all. However, sometimes the presence of a small amount of the said component is not avoidable e.g., due to impurities contained in the raw materials used. “Essentially free of” typically means a content of less than 1, 0.5 or 0.1 wt. %.

The invention is advantageous for a couple of reasons:

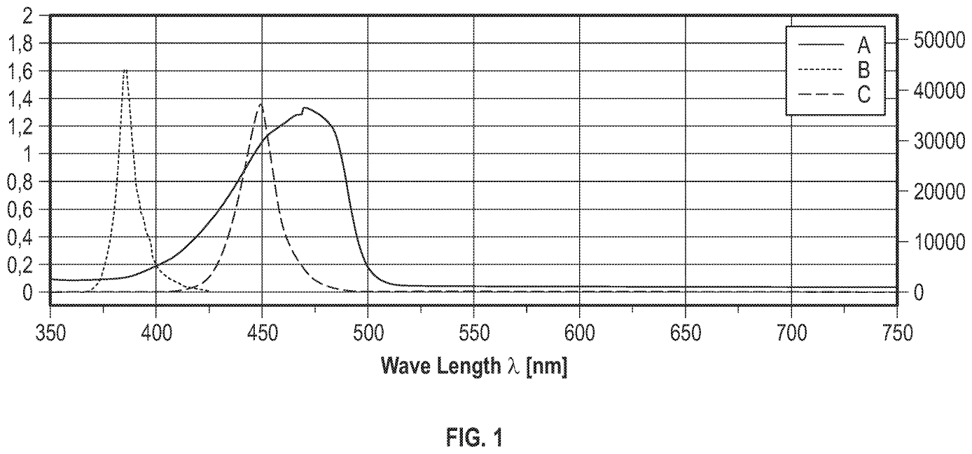

The photo-initiator described in the present text absorbs light not only in the region of visible light but also in the region of UV light. This is an advantage e.g., over the photo-initiator camphor quinone (CQ) which shows absorption mainly in the region of visible light as shown in FIG. 1.

In FIG. 1 the absorption spectrum of CQ is marked as A, the emission spectrum of a typical 3D-printing device as B and the emission spectrum of a typical dental curing light device as C.

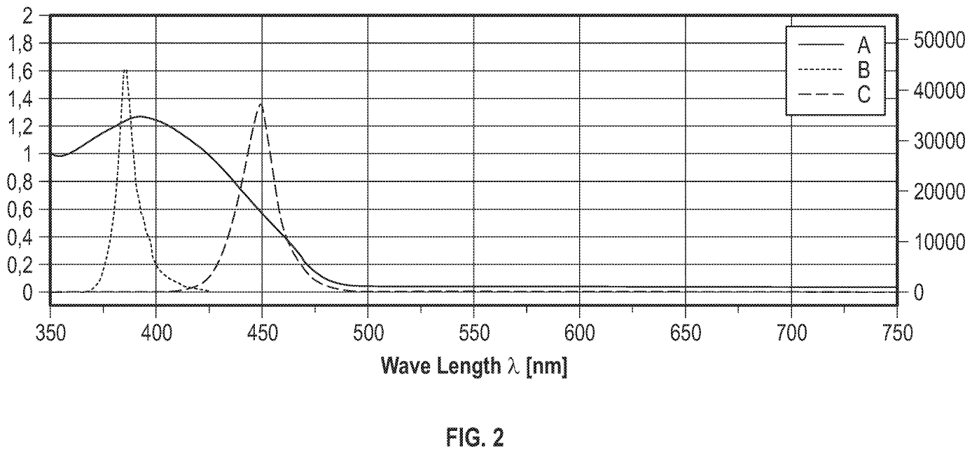

The photo-initiator described in the present text is not only sensitive to light which is typically used in additive-manufacturing devices but also sensitive to light which is typically used in dental curing lights. For the photo-initiator phenyl-1,2-propanedione (PPD) this is shown in FIG. 2.

In FIG. 2 the absorption spectrum of PPD is marked as A, the emission spectrum of a typical 3D-printing device as B and the emission spectrum of a typical dental curing light device as C.

Further, the absorption of the photo-initiator in the UV light region is sufficiently high enough for obtaining a pre-cured article having appropriate mechanical properties, in particular high flexibility.

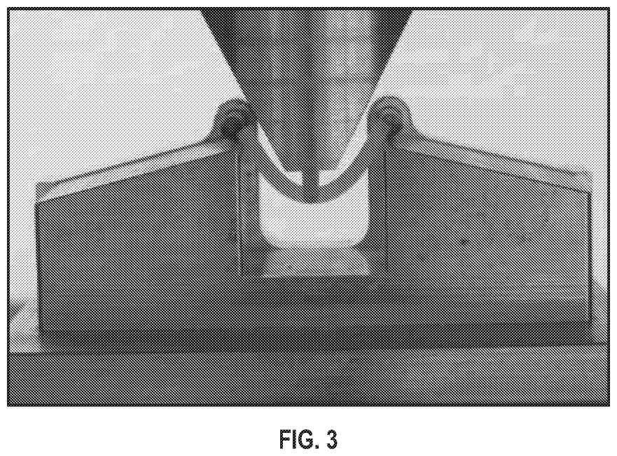

The flexibility of the pre-cured article might even be so high that a typical determination of flexural strength is not possible as the test bar used for measuring does not break. So, the pre-cured article might be regarded as fully elastic or rubber-elastic. This is shown in FIG. 3.

However, the obtained pre-cured article still contains sufficiently high amounts of uncured moieties and photo-initiator allowing the pre-cured article not only to be cured at a later stage, but also offering the option of adhesively fixing the pre-cured article to hard dental tissue or offering the option of adhesively fixing the pre-cured article to other dental materials, wherein the adhesive fixation is triggered by a light-induced curing step in the visible light region.

The photo-initiator described in the present text is also favorable from an aesthetic point of view as it is essentially colorless in the visible light region or has only a slight yellow color. So, the color of a dental or orthodontic article obtained by radiation-curing a curable composition containing this photo-initiator is not negatively affected or influenced and does not show an undesired discoloration after curing.

As the photo-initiator is essentially colorless, the photo-initiators can also be used in a comparable high amount without negatively affecting the aesthetic properties. This allows for a high process flexibility during the additive-manufacturing process and also enables a high conversion rate of the curable moieties, if desired.

Further, as the photo-initiator shows a high absorption and sensitivity in the UV light region, the curing reaction during the additive-manufacturing process proceeds fast which allows an economic production of the pre-cured articles.

These properties are especially useful in dental or orthodontic procedures, that require an extraoral pre-cure and a final intraoral post-cure step, e.g. for producing pre-formed dental composite crowns, orthodontic attachments for clear tray aligners and/or orthodontic brackets.

The curable composition described in the present text is for use in a process of treating a dental situation in the mouth of a patient.

The curable composition comprises one or more radiation-curable components, a photo-initiator for curing the radiation-curable components, optionally filler(s) and optionally additive(s).

The curable composition described in the present text can be characterized as one-part light-curable composition.

The curable composition can be further characterized by the following features alone or in combination:
a. viscosity: <50 Pa*s at 23° C. and a shear rate of 1 s⁻¹; or within a range of 1 to less than 40 Pa*s at 23° C. a shear rate of 1 s⁻¹;
b. curable by radiation having a wavelength in the range of 350 to 500 nm.

A viscosity of the curable composition in the above range was found to be particular suitable for processing the curable composition in an additive-manufacturing process.

The radiation-curable component is typically a component comprising one or more ethylenically unsaturated moieties.

The radiation-curable components can be selected from (meth)acrylate components, urethane (meth)acrylate components and mixtures thereof. Using a mixture of (meth)acrylate components, urethane (meth)acrylate components is sometimes preferred.

The radiation-curable component(s) are typically present in the following amounts: at least 20, or at least 25, or at least 30 wt. %; at most 95, or at most 90, or at most 80 wt. %; from 20 to 95, or 25 to 90, or 30 to 80 wt. %; wt. % with respect to the curable composition.

The curable composition may comprise one or more (meth)acrylate components not comprising a urethane moiety.

The (meth)acrylate(s) not comprising a urethane moiety is different from a urethane (meth)acrylate, e.g., with respect to functionality, chemical moieties, molecular weight or combinations thereof.

The (meth)acrylate components not comprising a urethane moiety can typically be characterized by the following properties alone or in combination: a) comprising at least 2 (meth)acrylate moieties; b) molecular weight: 170 to 1,000 g/mol.

Examples include di- or poly-acrylates and methacrylates such glycerol diacrylate, glycerol triacrylate, ethyleneglycol diacrylate, diethyleneglycol diacrylate, triethyleneglycol dimethacrylate, 1,3-propanediol diacrylate, 1,3-propanediol dimethacrylate, trimethylolpropane triacrylate, 1,2,4-butanetriol trimethacrylate, 1,4-cyclohexanediol diacrylate, pentaerythritol triacrylate, pentaerythritol tetraacrylate, pentaerythritol tetramethacrylate, sorbitol hexacrylate, bis[1-(2-acryloxy)]-p-ethoxyphenyldimethylmethane, bis[1-(3-acryloxy-2-hydroxy)]-p-propoxyphenyl-dimethylmethane; the bis-acrylates and bis-methacrylates of polyethylene glycols of molecular weight 200-500, copolymerizable mixtures of acrylated monomers. Suitable monomers are also described in U.S. Pat. No. 4,652,274 (Boettcher et al.) and U.S. Pat. No. 4,642,126 (Zador et al.), the content of which is herewith incorporated by reference.

Preferred ethylenically unsaturated monomers are methacrylate and acrylate monomers, such as di(meth)acrylates of propanediol, butanediol, hexanediol, octanediol, nonanediol, decane-diol and eicosanediol, di(meth)acrylates of ethylene glycol, of polyethylene glycols and of polypropylene glycols, di(meth)acrylates of ethoxylated bisphenol A, for example 2,2′-bis(4-(meth)acryloxytetraethoxyphenyl)propanes, and (meth)acrylamides. The monomers used can furthermore be esters of [alpha]-cyanoacrylic acid, crotonic acid, cinnamic acid and sorbic acid.

It is also possible to use methacrylic esters including those mentioned in U.S. Pat. No. 4,795,823 (Schmitt et al.), including bis[3[4]-methacryl-oxymethyl-8(9)-tricyclo[5.2.1.0^{2,6}]decylmethyl triglycolate. Suitable are also 2,2-bis-4(3-methacryloxy-2-hydroxypropoxy)phenylpropane (Bis-GMA), 2,2-bis-4(3-methacryloxypropoxy)phenylpropane, triethylene glycol dimethacrylate (TEGDMA), and di(meth)acrylates of bishydroxymethyltricyclo-(5.2.1.0^{2,6})decane.

If desired, the curable composition may also comprise (meth)acrylate components comprising only one (meth)acrylate moiety, such as e.g. methyl acrylate, methyl methacrylate, ethyl (meth)acrylate, isopropyl (meth)acrylate, n-hexyl (meth)acrylate, stearyl (meth)acrylate, allyl (meth)acrylate, glycerol di(meth)acrylate and mixtures thereof.

Suitable compounds also include 2-hydroxyethyl (meth)acrylate (HEMA), 2- or 3-hydroxypropyl (meth)acrylate, 4-hydroxybutyl (meth)acrylate, 5-hydroxypentyl (meth)acrylate, 6-hydroxyhexyl (meth)acrylate, 10-hydroxydecyl (meth)acrylate, dialkylene glycol mono(meth)acrylate, for example, diethylene glycol mono(meth)acrylate, triethylene glycol mono(meth)acrylate, tetraethylene glycol mono(meth)acrylate, polyethylene glycol mono(meth)acrylate, dipropylene glycol mono(meth)acrylate, polypropylene glycol mono(meth)acrylate, and further 1,2- or 1,3- and 2,3-dihydroxypropyl (meth)acrylate, 2-hydroxypropyl-1,3-di(meth)acrylate, 3-hydroxypropyl-1,2-di(meth)acrylate, N-(meth)acryloyl-1,2-dihydroxypropylamine, N-(meth)acryloyl-1,3-dihydroxypropylamine, adducts of phenol and glycidyl (meth)acrylate, for example, 1-phenoxy-2-hydroxypropyl (meth)acrylate, and 1-naphthoxy-2-hydroxypropyl (meth)acrylate. If desired, mixtures of one or more of these components can be used.

If present, and if present in combination with other polymerizable components such as urethane (meth)acrylate components, the (meth)acrylate components are typically present in the following amounts: at least 20, or at least 25, or at least 30 wt. %; at most 75, or at most 70, or at most 65 wt. %; from 20 to 75, or 25 to 70, or 30 to 65 wt. %; wt. % with respect to the curable composition.

The curable composition may also comprise one or more urethane (meth)acrylates.

The urethan (meth)acrylate typically comprises at least two (meth)acrylate moieties and at least two urethane moieties.

The molecular weight of the urethane(meth)acrylate is typically at least 400 g/mol or at least 800 g/mol or at least 1,000 g/mol.

Useful ranges include from 400 to 3,000 g/mol or from 800 to 2,700 g/mol or from 1,000 to 2,500 g/mol.

The urethane(meth)acrylates employed in the composition are typically obtained by reacting an NCO-terminated compound with a suitable monofunctional (meth)acrylate monomer such as hydroxyethyl acrylate, hydroxyethyl methacrylate, hydroxypropylmethacrylate, preferably hydroxyethyl- and hydroxypropylmethacrylate.

Urethane (meth)acrylates may be obtained by a number of processes known to the skilled person.

For example, a polyisocyanate and a polyol may be reacted to form an isocyanate-terminated urethane prepolymer that is subsequently reacted with a (meth)acrylate such as 2-hydroxy ethyl(meth)acrylate. These types of reactions may be conducted at room temperature or higher temperature, optionally in the presence of catalysts such as tin catalysts, tertiary amines and the like.

Polyisocyanates which can be employed to form isocyanate-functional urethane prepolymers can be any organic isocyanate having at least two free isocyanate groups. Included are aliphatic cycloaliphatic, aromatic and araliphatic isocyanates.

Any of the known polyisocyanates such as alkyl and alkylene polyisocyanates, cycloalkyl and cycloalkylene polyisocyanates, and combinations such as alkylene and cycloalkylene polyisocyanates can be employed.

Preferably, diisocyanates having the formula X(NCO)₂ are used, with X representing an aliphatic hydrocarbon radical with 2 to 12 C atoms, a cycloaliphatic hydrocarbon radical with 5 to 18 C atoms, an aromatic hydrocarbon radical with 6 to 16 C atoms and/or an araliphatic hydrocarbon radical with 7 to 15 C atoms.

Examples of suitable polyisocyanates include 2,2,4-trimethylhexamethylene-1,6-diisocyanate, hexamethylene-1,6-diisocyanate (HDI), cyclohexyl-1,4-diisocyanate, 4,4′methylene-bis(cyclohexyl isocyanate), 1,1′-methylenebis(4-isocyanato) cyclohexane, isophorone diisocyanate, 4,4′-methylene diphenyl diisocyanate, 1,4-tetramethylene diisocycanate, meta- and para-tetra-methylxylene diisocycanate, 1,4-phenylene diisocycanate, 2,6- and 2,4-toluene diisocycanate, 1,5-naphthylene diisocycanate, 2,4′ and 4,4′-diphenylmethane diisocycanate and mixtures thereof.

It is also possible to use higher-functional polyisocyanates known from polyurethane chemistry or else modified polyisocyanates, for example containing carbodiimide groups, allophanate groups, isocyanurate groups and/or biuret groups. Particularly preferred isocyanates are isophorone diisocyanate, 2,4,4-trimethyl-hexamethylene diisocyanate and higher-functional polyisocyanates with isocyanurate structure.

The isocyanate terminated urethane compound is capped with a (meth)acrylate to produce a urethane(meth)acrylate compound. In general, any (meth)acrylate-type capping agent having a terminal hydroxyl group and also having an acrylic or methacrylic moiety can be employed, with the methacrylic moiety being preferred.

Examples of suitable capping agents include 2-hydroxyethyl(meth)acrylate, 2-hydroxypropyl (meth)acrylate, glycerol di(meth)acrylate and/or trimethylolpropane di(meth)acrylate. Particularly preferred are 2-hydroxyethyl methacrylate (HEMA) and/or 2-hydroxyethyl acrylate (HEA).

The equivalence ratio of isocyanate groups to compounds reactive vis-á-vis isocyanate groups is 1.1:1 to 8:1, preferably 1.5:1 to 4:1.

The isocyanate polyaddition reaction can take place in the presence of catalysts known from polyurethane chemistry, for example organotin compounds such as dibutyltin dilaurate or amine catalysts such as diazabicyclo[2.2.2]octane. Furthermore, the synthesis can take place both in the melt or in a suitable solvent which can be added before or during the prepolymer preparation.

Suitable solvents are for example acetone, 2-butanone, tetrahydrofurane, dioxane, dimethylformamide, N-methyl-2-pyrrolidone (NMP), ethyl acetate, alkyl ethers of ethylene and propylene glycol and aromatic hydrocarbons. The use of ethyl acetate as solvent is particularly preferred.

Suitable examples of urethane (meth)acrylates include 7,7,9-trimethyl-4,13-dioxo-3,14-dioxa-5,12-diazahexadecane-1,16-dioxy-dimethacrylate (e.g., Plex™ 666-1, Rohm), urethane (meth)acrylates derived from 1,4 and 1,3-Bis(1-isocyanato-1-methylethyl)benzene (e.g. as described in EP 0 934 926 A1) and mixtures thereof.

According to one embodiment, the urethane(meth)acrylate is characterized as follows:
having the structure A-(-S1-U-S2-MA)ₙ, with
A being a connector element comprising at least one unit,
S1 being a spacergroup comprising at least 4 units connected with each other,
S2 being a spacergroup comprising at least 4 units connected with each other,
the units of A, S1 and S2 being independently selected from CH₃—, —CH₂—, —O—, —S—, —NR¹—, —CO—, —C—R¹═,

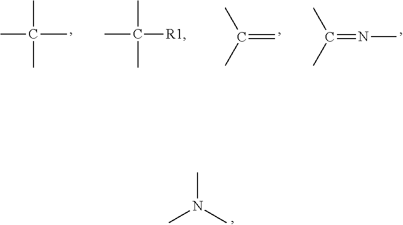

—N═, —CR¹R²—,
with R¹ and R² being independently selected from hydrogen, alkyl, substituted alkyl, alkenyl, cycloalkyl, substituted cycloalkyl, arylalkyl, aryl or substituted aryl, wherein these units can form linear, branched or cyclic structures such as alkyl, cycloalkyl, aryl, ester, urethane or amide groups,
U being a urethane group connecting spacergroups S1 and S2,
MA being an acrylate or methacrylate group and
n being 3 to 6.

According to one embodiment the urethane (meth)acrylate is represented by the structure

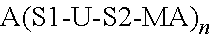

with

A being a connector element comprising at least 1, 2, 3, 4, 5, 6, 7, 8, 9, 10, 11, 12, 13, 14, 15, 16, 17, 18, 19 or 20 units,
S1 being a spacergroup comprised of units connected with each other and comprising at least 4, 5, 6, 7, 8, 9 or 10 units,
S2 being a spacergroup comprised of units connected with each other and comprising at least 4, 5, 6, 7, 8, 9, 10, 12, 15, 20 or 25 units,
U being a urethane group connecting spacergroups S1 and S2,
MA being an acrylate or methacrylate group and
n being 3 to 6 or 4 to 6 or 5 to 6.

It can be preferred, if A has a cyclic structure and comprises at least about 6 units.

It can further be preferred, if S1 has a linear or branched structure and comprises at least 4 or 6 units.

It can further be preferred, if S2 has a linear or branched structure and comprises at least 6 or 8 units.

A urethane(meth)acrylate wherein A has a cyclic structure and comprises at least 6 units and S1 has a linear structure and comprises at least 4 units and S2 has a linear structure and comprises at least 8 units and U is a urethane group can also be preferred.

Neither the atoms of the urethane group connecting S1 and S2 nor the atoms of the (meth)acrylgroup belong to the spacergroup S1 or S2. Thus, the atoms of the urethane group do not count as units of the spacergroups S1 or S2.

The nature and structure of the connector element is not particularly limited. The connector element can contain saturated (no double bonds) or unsaturated (at least one or two double bonds) units, aromatic or hetero aromatic units (aromatic structure containing atoms including N, O and S).

Specific examples of connector element A having a cyclic structure include:

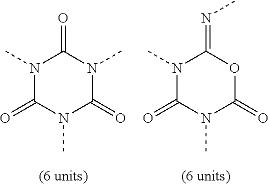

Specific examples of connector element A having a non-cyclic but branched structure include:

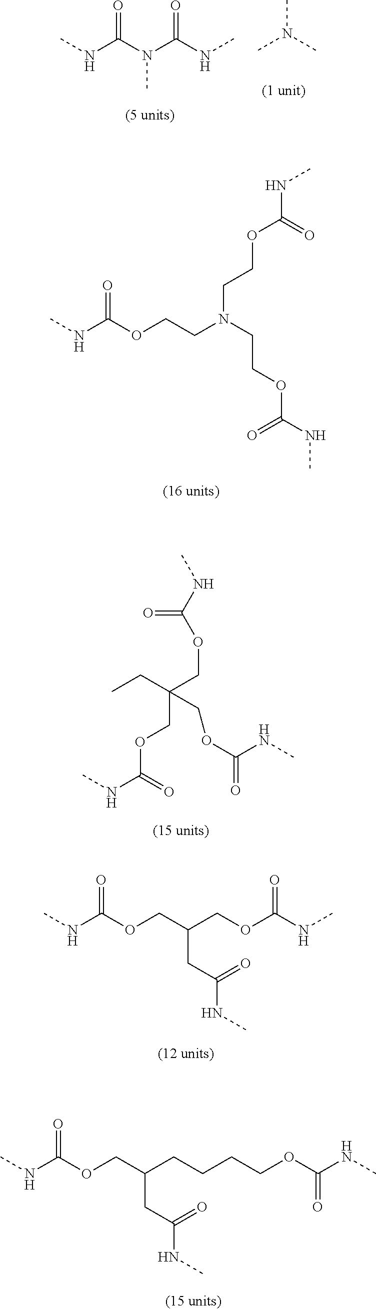

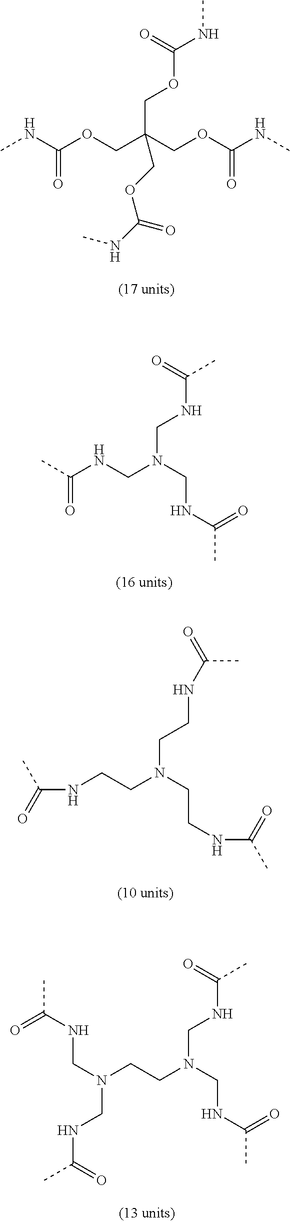

The dotted lines indicate the bondings to the spacergroup S1.

The nature and structure of the spacergroups S1 or S2 is not particularly limited, either.

The spacergroups are comprised of units connected with each other. Typical units include: CH₃—, —CH₂—, —O—, —S—, —NR¹—, —CO—, —CR¹═,

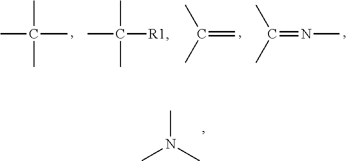

N, —N═, —CR¹R²—, with R¹ and R² independently selected from hydrogen, alkyl, substituted alkyl, alkenyl, cycloalkyl, substituted cycloalkyl, arylalkyl, aryl or substituted aryl.

These units can form linear, branched or cyclic structures such as alkyl, cycloalkyl, aryl, ester, urethane or amide groups.

The structure of S1 can be identical to the structure of S2. However, in some embodiments the structure of S1 is different from S2. In a specific embodiment the number of units being present in S1 is less or equal than the number of units being present in S2.

In a specific embodiment, S1 may have a saturated hydrocarbon structure.

In another specific embodiment, S2 may have a saturated hydrocarbon structure.

Typical examples of useful spacer groups for S1 include:

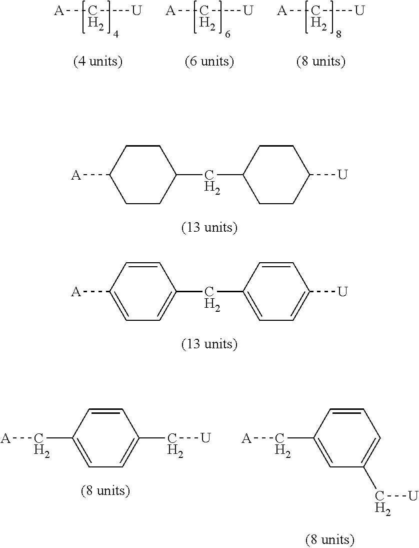

The dotted lines indicate the chemical bonding to either the group A or the group U.

Typical examples of useful spacer groups for S2 include:

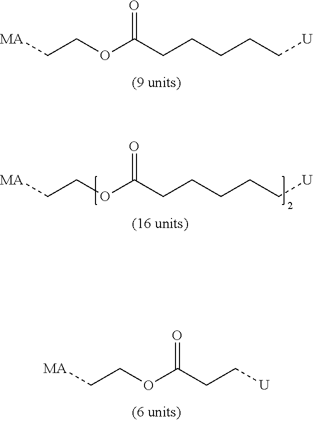

The dotted lines indicate the chemical bonding to either the (meth)acrylate group or the group U. The number of the units to be counted according to the invention is given in brackets.

Specific examples of the urethane (meth)acrylate include

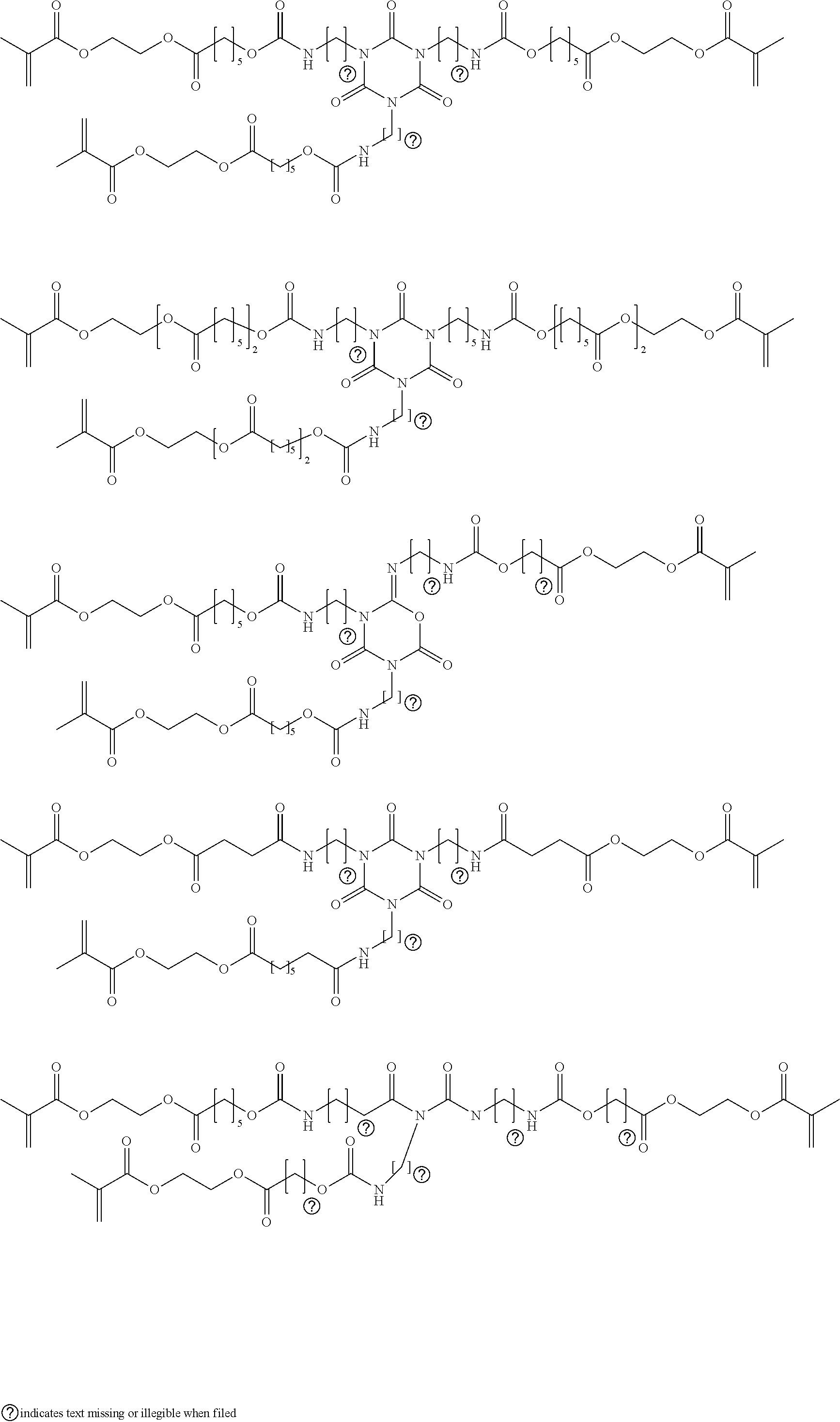

Further suitable urethan(meth)acrylates are based on alpha-omega-terminated poly(meth)acrylatdiols (e.g., as described in EP 1 242 493 B1) or can be a polyester, polyether, 5 polybutadiene or polycarbonate urethane(meth)acrylate (e.g., as described in U.S. Pat. No. 6,936,642 B2).

If present, the urethane (meth)acrylate is typically present in the following amounts: at least 5, or at least 8, or at least 10 wt. %; at most 30, or at most 25, or at most 20 wt. %; from 5 to 30, or 8 to 25, or 10 to 20 wt. %; wt. % with respect to the curable composition.

The (meth)acrylate not comprising a urethane moiety is typically used in excess over the (meth)acrylate comprising a urethane moiety by weight.

The ratio of ((meth)acrylate not comprising a urethane moiety)/((meth)acrylate comprising a urethane moiety) is typically in a range of 10/1 to 2/1 with respect to weight.

The curable composition also comprises one or more photo-initiators.

Suitable photo-initiators are those which are able to start or initiate a curing reaction of the radiation-curable components upon radiation. In this respect, the photo-initiators described in the present text are capable of generating free radicals upon exposure to radiation in the wavelength regions described in the present text, i.e. in the region of visible light and the region of UV light.

These photo-initiators are also referred to as multi-wavelengths photo-initiators.

The curable composition typically comprises only photo-initiator(s) having an absorption band in the UV light region and the visible light region.

According to one embodiment, the curable composition comprises only one photo-initiator.

The photo-initiator described in the present text has a special absorption behavior.

The absorption spectrum covers not only the region of UV light but also the region of visible light. Thus, the absorption spectrum comprises two regions, one for absorbing UV light and one for absorbing visible light, wherein the absorption in the UV light region is stronger than the absorption in the visible light region.

Stronger absorption means that at a given wavelength the absorption curve or absorption value obtained from a UV/VIS spectrometer is located above the absorption curve or absorption value at a different wavelength.

For the photo-initiators described in the present text the ratio of the absorption in the range of 350 to 410 nm, in particular at 390 nm, to the absorption in the range of 440 to 500 nm, in particular at 450 nm, is typically in a range of 1.05 to 13 or 1.10 to 10 or 1.15 to 5.

Further, certain embodiments of the photo-initiator are typically non-fluorescent.

The photo-initiator may comprise a di-ketone moiety, a titanocene moiety or an acylgermanium moiety.

Examples of suitable photo-initiators include components comprising a phenyl-1,2-propanedione (PPD) moiety, components comprising a benzil moiety, bis(cyclopentadienyl) bis [2,6-difluoro-3-(1-pyrryl)phenyl titanium (Omnirad™ 784), monoacyl or diacylgermanium moiety, and mixtures thereof.

The formulas of the respective moieties are given below:

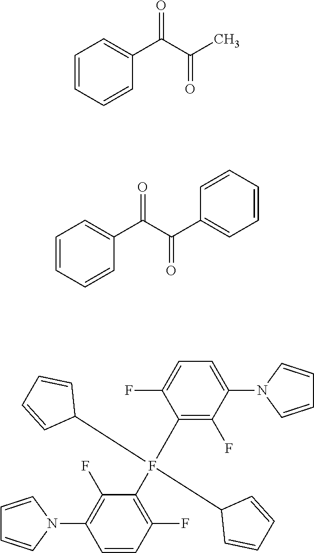

In particular components comprising a phenyl-1,2-propanedione moiety were found to be useful as 3D-articles obtained by radiation-curing of the respective curable composition show essentially no discoloration after the 2^{th} a curing step. Further, components comprising a phenyl-1,2-propanedione moiety are typically liquid which facilitates the mixing with the other components of the radiation curable composition.

The photo-initiator is often used in combination with an activator. As activator tertiary amines are often used.

Suitable examples of the tertiary amines include N,N-dimethyl-p-toluidine, N,N-dimethyl-aminoethyl methacrylate (DMAEMA), triethanolamine, methyl 4-dimethylaminobenzoate, ethyl 4-dimethylaminobenzoate (EDMAB), methyldiphenylamine, 4-(N,N-dimethylamion)phenethyl alcohol, (DMPOH) and isoamyl 4-dimethylaminobenzoate.

The photo-initiator optionally in combination with an activator is typically present in the following amounts: lower amount: at least 0.01, at least 0.02 or at least 0.03 wt. %; upper amount: at most 5, at most 4, or at most 3 wt. %; range: 0.01 to 5, or 0.02 to 4, or 0.03 to 3 wt. %, wt. % with respect to the curable composition.

The curable composition may also comprise one or more filler(s). The nature of the filler is not particularly limited unless the intended use cannot be obtained.

Suitable fillers include non acid-reactive glasses such as lanthanum glass, borosilicate glass, soda glass, barium glass, strontium glass, glass ceramic, aluminosilicate glass, barium boroaluminosilicate glass, strontium boroaluminosilicate glass; silicates such as calcium silicate, zirconium silicate; and metal oxides such as quartz, cristobalite, alumina, titania, silica-titania, silica-titania-barium oxide, silica-zirconia, silica-alumina and mixtures thereof.

Fillers which can also be used include fillers comprising discrete nano-sized filler particles, aggregated filler particles and mixtures thereof can be used.

Compositions containing nano-sized filler particles are typically more transparent than compositions containing larger filler particles.

The average particle size of the nano-sized filler particles is typically 40 nm and below, or 35 nm and below, or 30 nm and below. The average particle size is typically in a range of 10 to 40 nm or 10 to 35 or 10 to 30 nm.

The specific surface area (BET) of the nano-sized filler is preferably 80 m²/g or more, or 100 m²/g or more or 120 m²/g or more. The specific surface area (BET) is typically in a range of 80 to 500 m²/g or 100 to 400 m²/g or 120 to 300 m²/g.

If desired, the specific surface can be determined according to Brunauer, Emmet and Teller (BET) by using a device (Monosorb™) available from Quantachrome.

The nano-sized filler comprise, contain, consist essentially or consist of aggregated nano-sized particles. If desired, this can be proven by transmission electron microscopy (TEM).

The filler particles typically comprise oxides of S1, Zr, Al and mixtures thereof, wherein the oxides of S1 and Zr are sometimes preferred.

Suitable fumed silicas include for example, products sold under the tradename Aerosil™ series OX-50, -130, -150, and -200, Aerosil™ R8200, R805 available from Evonik, CAB—O-SIL™ M5 available from Cabot Corp (Tuscola), and HDK types e.g., HDK™—H2000, HDK™ H15, HDK™ H18, HDK™ H20 and HDK™ H30 available from Wacker.

Nano-sized silicas are also commercially available from Nalco Chemical Co. (Naperville, Ill.) under the product designation NALCO™ COLLOIDAL SILICAS. For example, preferred silica particles can be obtained from using NALCO™ products 1040, 1042, 1050, 1060, 2327 and 2329. Other suitable nano-sized silicas are commercially available from Covestro (Leverkusen, Germany) under the product designation Dispercoll™ (for example Dispercoll™ S 3030 or Dispercoll™ S 4020), Grace GmbH & Co. KG (Worms, Germany) under the product designation Ludox™ (for example Ludox™ P-X30 or Ludox™ P-W30) and Nouryon (Amsterdam, Netherlands) under the product designation Levasil™ (for example Levasil™ CS50-34P).

Aggregated filler particles typically comprise nano-clusters.

Compared to other fillers, using nano-cluster(s) can be beneficial because it allows the formulation of a composition with high filler load resulting in better mechanical properties, e.g. polishability or abrasion and higher aesthetics.

A suitable nano-filler comprising aggregated nano-sized particles can be produced according to the processes described e.g., in preparatory example A and B of U.S. Pat. No. 6,730,156 (Windisch et. al).

Once dispersed in the resin, the filler particles remain in an aggregated stage. That is, during the dispersion step the particles do not break up into discrete (i.e., individual) and un-associated (i.e., non-aggregated) particles.

The nano-sized filler particles are typically surface treated.

Surface treating allows an easier dispersing of the nano-sized filler particles in the monomer matrix and may prevent settling of the fillers from the formulation during storage.

Useful surface treatment agents include silanes.

The silane surface treating agents can comprise a polymerizable moiety or may not comprise a polymerizable moiety, in particular a (meth)acrylate moiety. Only one silane surface treating agent or mixtures of different silane treating agents can be used.

In a specific embodiment a mixture of a silane surface treating agent comprising a polymerizable moiety, in particular a (meth)acrylate moiety, and a silane surface treating agent not comprising a polymerizable moiety is used.

If the surface treating is done with two different silane surface treating agents, the polymerizable silane surface treating agent is typically used in a higher amount with respect to weight compared to the non-polymerizable silane surface treating agent.

A ratio of the polymerizable silane surface treating agent to the non-polymerizable silane surface treating agent in the range of 90/10 to 60/40 or 80/20 to 70/30 with respect to weight was found to be useful.

If desired, the surface of the treated particles can be analysed using FT-IR or NMR technologies.

The polymerizable silane surface treating agent is usually an alkoxy silane, preferably a trialkoxy silane comprising a (meth)acrylate group.

Typical embodiments can be characterized by the following formula:

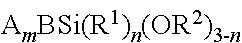

with A comprising a (meth)acryl moiety,
B comprising a spacer group, such as (i) linear or branched C₁ to C₁₂ alkyl, (ii) C₆ to C₁₂ aryl, (iii) organic group having 2 to 20 carbon atoms bonded to one another by one or more ether, thioether, ester, thioester, thiocarbonyl, amide, urethane, carbonyl and/or sulfonyl linkages,
R¹ comprising an alkyl group (e.g., C₁ to C₆) or an aryl group (e.g., C₆ to C₁₂), and
R² comprising an alkyl group (e.g., C₁ to C₆),
with m=1, 2, or 3, and n=0, 1 or 2.

Examples of (meth)acrylate functionalized trialkoxy silanes include, but are not limited to 3-(meth)acryloxypropyl trimethoxysilane, 3-(meth)acryloxypropyl triethoxysilane, 3-(meth)acryl-oxypropyl tris(methoxyethoxy)silane, 3-(meth)acryloxypropenyl trimethoxysilane, (meth)acryloxy-ethyldimethyl(3-trimethoxysilylpropyl)ammonium chloride, N-(3-(meth)acryloxy-2-hydroxy-propyl)-3-aminopropyltriethoxysilane, O-((meth)acryloxyethyl)-N-(triethoxysilylpropyl)urethane, (meth)acryloxymethyl trimethoxysilane, (meth)acryloxymethyl triethoxysilane, (meth)acryloxymethyl methyldimethoxysilane, (meth)acryloxymethyl methyldiethoxysilane, (meth)acryloxyoctyl trimethoxysilane, [(meth)acryloxymethyl]phenethyl trimethoxysilane, 0-[(meth)acryloxyethyl]-N-(triethoxysilylpropyl)carbamate, (meth)acryloxypropyl triisopropoxy-silane, (meth)acryloxypropyl methyldimethoxysilane, (meth)acryloxypropyl methyldiethoxysilane, 3-(meth)acryloxypropyl dimethylmethoxysilane, 3-(meth)acryloxypropyl dimethylethoxysilane, (meth)acryloxymethyl dimethylmethoxysilane, (meth)acryloxymethyl dimethylethoxysilane, oligomeric hydrolysate of 3-(meth)acryloxypropyl trimethoxysilane, oligomeric hydrolysate of 3-(meth)acryloxypropyl triethoxysilane.

The non-polymerizable silane surface treating agent is usually an alkoxy silane, preferably a trialkoxy silane.

Typical embodiments can be characterized by the following formula:

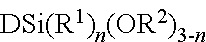

with D comprising (i) a linear or branched non-substituted or substituted (e.g., with one or more amino or mercapto groups) C₁ to C₁₆ alkyl, (ii) a non-substituted or substituted (e.g., with one or more amino or mercapto groups) C₆ to C₁₂ aryl group or (iii) an organic group having 2 to 20 carbon atoms bonded to one another by one or more ether, thioether, ester, thioester, thiocarbonyl, amide, urethane, carbonyl and/or sulfonyl linkages,
R¹ comprising an alkyl group (e.g., C₁ to C₆) or an aryl group (e.g., C6 to C₁₂), and
R² comprising an alkyl group (e.g., C₁ to C₆),
with n=0, 1 or 2.

Suitable non-polymerizable silane surface treating agents include phenyltrimethoxy silane, phenyltriethoxy silane, octyltrimethoxy silane, octyltriethoxy silane, hexadecyltrimethoxy silane, isobutyltrimethoxy silane, isobutyltriethoxy silane, propyltrimethoxy silane, 3-aminopropyl-methyldiethoxy silane, 3-aminopropyltrimethoxy silane, 3-mercaptopropyltrimethoxy silane, N-(2-aminoethyl)-3-aminopropyltrimethoxy silane, N-cyclohexyl-3-aminopropyltrimethoxy silane, 3-ureidopropyltrimethoxy silane, (cyclohexyl)methyldimethoxy silane and mixtures thereof.

Polymerizable and non-polymerizable silane surface treating agents are commercially available e.g. from Wacker (Minchen, Germany) under the product designation Geniosil™ or from Evonik (Hanau, Germany) under the product designation Dynasylan™ The process for surface treating discrete nano-sized filler particles typically comprises the following steps:
mixing a sol containing nano-sized particles with a silane surface treating agent; and
stirring the mixture under reflux in solvent like ethanol for several hours (e.g., 2 to 10 h);
after stirring for several hours (e.g., 2 to 10 h) undermixing of monomer into resulting mixture and stirring again for several hours (e.g., 2 to 10 h);
removing solvent under vacuum.

A suitable process is also described in U.S. Pat. No. 6,899,948 (Zhang et al.)

If present filler(s), in particular nano-sized filler particles, are typically present in the following amounts: at least 10, or at least 15, or at least 20 wt. %; or at most 70, or at most 60, or at most 50 wt. %; or from 10 to 70, or 15 to 60, or 20 to 50 wt. %; wt. % with respect to the curable composition.

Using such amounts of fillers typically contributes to the physical-mechanical properties of the composition, in particular in its cured state.

The curable composition typically also comprises one or more additives.

Additives which can be present include stabilizers, fluorescent dyes, UV light absorbers, fluoride release agents and mixtures thereof.

Suitable stabilizers include free radical scavengers such as substituted and/or unsubstituted hydroxyaromatics (e.g., butylated hydroxytoluene (BHT), hydroquinone, hydroquinone monomethyl ether (MEHQ), 3,5-di-tert-butyl-4-hydroxyanisole (2,6-di-tert-butyl-4-ethoxyphenol), 2,6-di-tert-butyl-4-(dimethylamino)methylphenol or 2,5-di-tert-butyl hydroquinone, 2-(2′-hydroxy-5′-methylphenyl)-2H-benzotriazole, 2-(2′-hydroxy-5′-t-octylphenyl)-2H-benzotriazole, 2-hydroxy-4-methoxybenzophenone (UV-9), 2-(2′-hydroxy-4′,6′-di-tert-pentylphenyl)-2H-benzo-triazole, 2-hydroxy-4-n-octoxybenzophenone, 2-(2′-hydroxy-5′-methacryloxyethylphenyl)-2H-benzotriazole, phenothiazine, and HALS (hindered amine light stabilizers).

Suitable fluorescent dyes often include an anthracene or perylene moiety. Fluorescent dyes typically have an absorption peak in the range of 350 to 450 nm. Commercially available fluorescent dyes include e.g., Lumilux™ Blau LZ, Lumilux™ Gelb LZ, and dyes comprising an anthracene moiety (e.g., 2-Ethyl-9,10-dimethoxyanthracene; EDMO).

If present, fluorescent dyes are typically present in an amount of 0.001 to 0.5 wt. % with respect to the weight of the composition.

Suitable UV light absorbers include components comprising a benzotriazole moiety. UV absorbers typically have an absorption peak in the range of 350 to 420 nm. Commercially available UV light absorbers include Tinuvin™ 326, Tinuvin™ 328, Tinuvin™ P, Uvinul™ M40.

If present, UV light absorbers are typically present in an amount of 0.001 to 1.0 wt. % with respect to the weight of the composition.

Examples of fluoride release agents include naturally occurring or synthetic fluoride minerals. These fluoride sources can optionally be treated with surface treatment agents.

Additives are typically present in the following amounts: 0 or at least 0.01 or at least 0.1 wt. %; or utmost 10 or 7.5 or 5 wt. %; or from 0 to 10 or 0.01 to 7.5 or 0.1 to 5 wt. %; wt. % with respect to the curable composition.

The curable composition described in the present text typically comprises, essentially consist of, or consist of the respective components in the following amounts:
radiation-curable component(s): 20 to 95 wt. %,
photo-initiator optionally in combination with an activator: 0.01 to 5 wt. %,
filler: 0 to 70 wt. %,
additives: 0 to 10 wt. %,
wt. % with respect to the curable composition, wherein the components above are those described in the present text.

The curable composition may also comprise, essentially consist of, or consist of the respective components also in the following amounts:
methacrylate not comprising a urethane moiety: 20 to 75 wt. %,
urethane (meth)acrylates: 5 to 30 wt. %,
photo-initiator optionally in combination with an activator: 0.01 to 5 wt. %,
filler: 10 to 60 wt. %,
additives: 0.01 to 7.5 wt. %,
wt. % with respect to the curable composition, wherein the components above are those described in the present text.

The curable composition may also comprise, essentially consist of, or consist of the respective components also in the following amounts:
methacrylate not comprising a urethane moiety: 30 to 65 wt. %,
urethane (meth)acrylates: 10 to 20 wt. %,
photo-initiator optionally in combination with an activator: 0.02 to 4 wt. %,
filler: 15 to 50 wt. %,
additives: 0.1 to 5 wt. %,
wt. % with respect to the curable composition, wherein the components above are those described in the present text.

More specific embodiments are given below:

### Embodiment 1

A curable composition for use in a process of treating a dental situation in the mouth of a patient,
the curable composition comprising, consisting essentially of, or consisting of radiation-curable component(s) selected from (meth)acrylate components, urethane (meth)acrylate components and mixtures thereof,
photo-initiator(s), selected from components comprising a phenyl-1,2-propanedione moiety, components comprising a benzil moiety, bis(Cyclopentadienyl) bis [2,6-difluoro-3-(1-pyrryl)phenyl titanium], and mixtures thereof,
optionally filler(s),
optionally additive(s),
the process comprising the steps of
additive manufacturing a dental or orthodontic article layer-by-layer using radiation with a wavelength in the UV light region,
attaching the dental or orthodontic article to a surface of hard dental tissue or a dental material,
applying radiation with a wavelength in the visible light region to the dental or orthodontic article.

### Embodiment 2

A curable composition for use in a process of treating a dental situation in the mouth of a patient,
the curable composition comprising, consisting essentially of, or consisting of radiation-curable component(s) selected from (meth)acrylate components, urethane (meth)acrylate components and mixtures thereof, in an amount of 20 to 95 wt. %, photo-initiator(s), selected from components comprising a phenyl-1,2-propanedione moiety, components comprising a benzil moiety, bis(Cyclopentadienyl) bis [2,6-difluoro-3-(1-pyrryl)phenyl titanium, and mixtures thereof,
optionally in combination with an activator,
filler(s) in an amount of 1 to 70 wt. %,
additive(s),
the process comprising the steps of
additive manufacturing a dental or orthodontic article layer-by-layer using radiation with a wavelength in the UV light region,
attaching the dental or orthodontic article to a surface of hard dental tissue,
applying radiation with a wavelength in the visible light region to the dental or orthodontic article.
wt. % with respect to the curable composition.

### Embodiment 3

A curable composition for use in a process of treating a dental situation in the mouth of a patient, the curable composition comprising, consisting essentially of or consisting of
radiation-curable component(s) selected from (meth)acrylate components, urethane (meth)acrylate components and mixtures thereof, in an amount of 20 to 75 wt. %,
photo-initiator(s), selected from components comprising a phenyl-1,2-propanedione moiety, components comprising a benzil moiety, bis(Cyclopentadienyl) bis [2,6-difluoro-3-(1-pyrryl)phenyl titanium, and mixtures thereof,
optionally in combination with an activator,
filler(s) in an amount of 10 to 50 wt. %,
additive(s),
the process comprising the steps of
additive manufacturing a dental or orthodontic article layer-by-layer using radiation with a wavelength in the UV light region,
applying a dental adhesive or dental cement to a surface section of the dental or orthodontic article intended to be attached to hard dental tissue,
attaching the dental or orthodontic article to a surface of hard dental tissue,
applying radiation with a wavelength in the visible light region to the dental or orthodontic article,
wt. % with respect to the curable composition.

### Embodiment 4

A curable composition for use in a process of treating a dental situation in the mouth of a patient, the curable composition comprising, consisting essentially of or consisting of
radiation-curable component(s) selected from (meth)acrylate components, urethane (meth)acrylate components and mixtures thereof, in an amount of 20 to 75 wt. %, photo-initiator(s) selected from components comprising a phenyl-1,2-propanedione moiety,
optionally in combination with an activator,
filler(s) in an amount of 10 to 50 wt. %,
additive(s),
the process comprising the steps of
additive manufacturing a dental or orthodontic article layer-by-layer using radiation with a wavelength in the UV light region,
applying a dental adhesive or dental cement to a surface section of the dental or orthodontic article intended to be attached to hard dental tissue,
attaching the dental or orthodontic article to a surface of hard dental tissue,
applying radiation with a wavelength in the visible light region to the dental or orthodontic article,
wt. % with respect to the curable composition.

The curable composition described in the present text can be produced by mixing the respective components under save-light conditions. If desired, a speed mixer can be used.

During storage the curable composition described in the present text is typically stored under save light conditions, in particular in a sealed container, vessel, or foil bag. The volume of the container may be in a range of 1 ml to 10 l or 5 ml to 5 l or 100 ml to 2 l.

The curable composition can be processed in an additive-manufacturing process for producing 3D-printed articles. The 3D-printing process as such is generally known to the skilled person.

An example of this kind of technology is described in U.S. Pat. No. 8,003,040 B2 (El-Siblani) relating to a process for producing a 3-dim object by solidifying layers with electromagnetic radiation of synergistic stimulation in a pattern.

In particular so-called SLA or DLP 3D-priting processes were found to be useful. Technical equipment which can be used is commercially available e.g., from companies like 3Shape, Rapid Shape, Formlabs, Lithoz, Prodways, Stratasys, EnvisionTec and others.

The additive-manufacturing device works with a certain radiation wavelength, which is typically in the range of 350 to 500 nm.

The additive-manufacturing device can also be characterized by the resolution which can be achieved. A suitable resolution is typically in the range of 5 to 100 μm or 10 to 80 μm or 20 to 60 μm.

After conducting the additive-manufacturing process, the printed article can be post-processed, if desired.

Useful post-processing steps include cleaning and optionally post-curing of the cleaned article.

The cleaning of the 3D-printed article can be done by using a cleaning solution and/or by conducting a so-called spin-cleaning process.

By conducting a cleaning step undesired residues of the curable resin remaining on the surface of the 3D-printed article can be removed.

Suitable cleaning solutions include alcohols such as ethanol or iso-propanol, esters of carboxylic acids such as di basic esters of a carboxylic acid and/or tri basic esters of a carboxylic acid, or mixtures thereof. Suitable cleaning solutions are also described in WO 2018/222395 A1 (3M).

The spin-cleaning process includes the step of moving or rotating the 3-dimensional article.

By doing this a mass inertial force is generated.

The term “mass inertial force” as referred to herein may be specified as force per unit mass and therefore may be specified in the unit m/s². Further, the mass inertial force can be expressed by the G-force which is a factor of the acceleration of gravity. For the purpose of the present text the acceleration of gravity is 9.81 m/s². Consequently, for example amass inertial force of 9.81 m/s² can be expressed as 1 G.

The acceleration force or mass inertial force is induced by moving, for example rotating, the object.

The centrifugal force on a particle on the surface of the 3-dim article typically depends on the rotation speed and the radius at which that particle is located from the rotation axis.

By varying parameters such as speed of movement or rotation, the duration thereof and/or the rotation axis, this technique allows the adjustment of the amount and layer-thickness of the radiation-curable composition remaining on the surface of the 3-dimensional article.

In an embodiment the mass inertial force corresponds to a G-force of at least 100 G. A mass inertial force of 100 G has proven to be suitable to remove a mid to high viscos radiation-curable material. The skilled person will recognize that the mass inertial force required for the cleaning may be lower for lower viscos materials and higher for higher viscos materials. Such a process is described e.g., in WO 2019/023120 A1 (3M).

For additive-manufacturing the dental or orthodontic article described in the present text, 3D-printing equipment which is commercially available can be used.

The use of the photo-initiator described in the present text allows for a curing of the radiation-curable composition, in particular if the following processing parameters are applied: layer thickness: 10 to 50 μm; wavelength of curing light: 350 to 420 nm; curing light intensity: 5 to 100 W/m²; exposure time of radiation: 1 to 20 s.

After having conducted the additive-manufacturing step, a pre-cured dental or orthodontic article is obtained. As no post-curing step has been applied, the pre-cured article still contains non-polymerized unsaturated moieties such as (meth)acrylate moieties, typically on its surface.

If desired, the presence and optionally the amount of non-polymerized (meth)acrylate functionalities can be characterized by determining the degree of conversion or alternatively by IR spectroscopy (e.g., Raman spectroscopy).

The pre-cured dental or orthodontic article can typically be characterized by the following features alone or in combination: a) being rubber-elastic; b) having an elongation at break in the range of 10 to 200%, if determined according to DIN EN ISO 527-1:2012-06.

The cured dental or orthodontic article can typically be characterized by the following features alone or in combination: a) having a flexural strength in the range of 60 to 200 MPa, if determined according to ISO/DIN 4049(2019); b) having an elongation at break in the range of 1 to 40%, if determined according to DIN EN ISO 527-1:2012-06.

The dental or orthodontic article, either in its pre-cured or cured stage, may have different shapes. The volume of the dental or orthodontic article is typically within a range of 0.1 to 10 ml or 0.2 to 5 ml.

E.g., a dental article may have the shape of a dental crown, dental bridge, dental onlay, dental inlay, dental veneer.

The curable composition described in the present text is in particular useful for producing dental articles having the shape of a dental crown, in particular for dental articles having the shape of a dental crown for pediatric use.

These kinds of dental composite crowns are described e.g., in U.S. Pat. No. 10,610,330 B2 (Herrmann et al) or US 2020/0206092 A1 (Herrmann et al.) The contents of these references are herewith incorporated by reference.

Dental composite crowns obtained by processing the curable composition of the present text in an additive-manufacturing process are typically ductile and can be shaped or adapted by the practitioner before or during the step of attaching the dental composite crown to the surface of a tooth stump in the mouth of patient, if desired.

Further, due to its elastic properties, the pre-formed dental composite crown can be easily placed on a tooth stump, even if there are undercuts.

The final curing or hardening step of the dental composite crown can then be done later, e.g., after the dental composite crown has been placed on a tooth stump in the mouth of a patient using a dental curing light with a wavelength in the region of visible light.

This provides the practitioner with more flexibility.

The orthodontic article may have the shape of an orthodontic attachment, orthodontic brackets.

Orthodontic article which can be produced by using the curable composition described in the present text are e.g., described in WO 2021/130624 A1 (3M), WO 2022/149083 (3M) or WO 2022/149084 A1 (3M). The contents of these references are herewith incorporated by reference.

The invention also relates to a kit of parts.

The kit of parts comprises, essentially consists of, or consists of the curable composition described in the present text, and a dental adhesive, dental cement, or dental primer and optionally a curing light, optionally a dental positioning tray, and optionally an instruction of use.

Dental adhesives are typically acidic dental composition with a rather low viscosity (e.g., 0.01 to 3 Pa*s at 23° C.). Dental adhesives directly interact with the enamel or dentin surface of a tooth. Dental adhesives are typically one-part compositions, are radiation-curable and comprise ethylenically unsaturated component(s) with acidic moiety, ethylenically unsaturated component(s) without acidic moiety, water, sensitizing agent(s), reducing agent(s) and additive(s).

Examples of dental adhesives are described in US 2020/0069532 A1 (Thalacker et al.) and US 2017/0065495 A1 (Eckert et al.). Dental adhesives are also commercially available, e.g. 3M™ Scotchbond™ Universal or 3M™ Scotchbond™ Universal Plus (3M Oral Care).

Suitable dental primers are described in U.S. Pat. No. 6,126,922 (Rozzi et al.) and WO 00/69393 A1 (3M). Dental primers are also commercially available, e.g., 3M™ Transbond™ XT Primer (3M Oral Care).

Dental cements which can be added to the kit include in particular self-adhesive resin cements, which contain an acidic polymerizable component (e.g., a (meth)acrylate component bearing a phosphoric or carboxylic acid moiety), polymerizable components without an acidic moiety, an initiator system and filler.

Suitable dental cements are also commercially available, such as RelyX™ Unicem 2, RelyX™ Universal or RelyX™ Luting Plus (3M Oral Care).

A dental positioning tray is typically used for placing orthodontic attachments onto patients' teeth. An example of a dental positioning tray and related processes are described in US 2015/0313687 A1 (Blees et al.), US 2020/131356 A1 (Zech et al.)

Suitable dental curing lights are described in U.S. Pat. No. 10,758,126 B2 (Geldmacher et al.) or U.S. Pat. No. 10,231,810 B2 (Gramann et al). Dental curing lights are also commercially available, e.g., 3M™ Elipar™ S10 or 3M™ Elipar™ Deep-Cure S LED curing light (3M Oral Care).

An instruction for use describes how the dental product or curable composition should be used in daily practice, e.g. outlining the application steps and optional curing conditions.

The curable composition is for use in a process of treating a dental situation in the mouth of a patient.

As described above, a pre-cured dental or orthodontic article or dental or orthodontic article comprising a pre-cured composition is produced first.

If desired, this pre-cured article can be post-processed, in particular cleaned.

The pre-cured article is then attached to a surface of hard dental tissue or another dental material. If desired, this process can be supported by using a dental adhesive or dental cement.

Thus, the process may comprise the additional steps of optionally post-processing the pre-cured dental or orthodontic article; and applying a dental adhesive or dental cement to a surface of the dental or orthodontic article which is intended to be attached to hard dental tissue or a dental material.

In a further step, the pre-cured article is typically radiation-cured using wavelength in the visible light region.

If the dental adhesive or dental cement is radiation-curable as well, the radiation curing of the pre-cured dental or orthodontic article and the dental adhesive or dental cement can be done simultaneously.

More particularly, a suitable process may comprise the following steps:
a. processing the curable composition in an additive-manufacturing process, optionally followed by post-processing steps such as cleaning, to obtain a 3D-printed pre-cured orthodontic article,
b. inserting the 3D-printed pre-cured orthodontic article into a cavity of a dental positioning tray,
c. applying a dental adhesive or a dental cement to the surface of the 3D-printed pre-cured orthodontic article which is intended to be attached to the surface of a tooth,
d. inserting the transparent dental positioning tray into the mouth of a patient,
e. radiation-curing the 3D-printed pre-cured orthodontic article,
f. removing the dental positioning tray from the mouth of the patient,
g. optionally inserting a dental aligner tray in engagement with the orthodontic article.

For an effective radiation-curing of the 3D-printed pre-cured orthodontic article, the dental positioning tray should be transparent to the light used for the radiation curing.

A dental aligner tray is used to straighten teeth like braces. They use gentle and constant force to move teeth in the desired position. They are typically transparent and custom made.

The process does typically not comprise the additional steps of roughening the surface of the 3D-printed dental or orthodontic article intended to be attached to hard dental tissue or the dental material; and/or applying a further radiation-curing step to the pre-cured dental or orthodontic article before the article is attached to the hard dental tissue or the dental material.

As the pre-cured article contains sufficiently large amounts of polymerizable moieties which are available for a copolymerization reaction with other (meth)acrylate components which are present e.g., in a dental adhesive or dental cement, the roughening of the surface of the pre-cured article for increasing the size of the surface is not needed.

For the same reason, the application of a further radiation-curing or post-curing step before the article is attached to hard dental tissue or another dental material, would be contra-productive as the amounts of polymerizable moieties would be reduced.

Further, the radiation-curable composition of the present text does typically not comprise the following components:
a) peroxide components in an amount of 0.1 or 0.3 or more wt. % with respect to the weight of the radiation-curable composition,
b) (meth)acrylates with acidic moieties in an amount of 2 or more wt. %, wt. % with respect to the weight of the radiation-curable composition.

Thus, the radiation-curable composition is essentially free of peroxide components or (meth)acrylates with acidic moieties and does not comprise peroxide components or (meth)acrylates with acidic moieties which have willfully been added.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. The above specification, examples and data provide a description of the manufacture and use of the compositions and methods of the invention. The invention is not limited to the embodiments disclosed herein. One skilled in the art will appreciate that many alternative embodiments of the invention can be made without departing from the spirit and scope of thereof.

The following examples are given to illustrate the invention.

### EXAMPLES

### Methods

### Viscosity

If desired, the viscosity can be measured with a Physica MCR 301 (Anton Paar Germany GmbH, Ostfildern-Scharnhausen) at 23.0° C. with a shear ramp between 0.1 s⁻¹ and of 1,000 s⁻¹ with a 25 mm plate/cone system.

### Method for Determining Light Absorption Bands

If desired, the light absorption spectrum can be determined by using a spectrophotometer Spectramax 190 (available from Molecular Devices LLC., Sunnyvale, CA, USA). As cuvette a multicuvette is used like Microtest 96-Well 370 μl Clear Plate (available from BD Biosciences Franklin Lakes, NJ, USA). 200 μl of solutions (photo initiator dissolved in TEGDMA) are placed into one of the 96 cuvettes and put into the Spectromax 190. The spectrum is recorded between 200 nm and 800 nm in 1 nm steps.

### Particle Size Distribution (Non Nano-Sized Particles)

If desired, the particle size can be measured using a Malvern Mastersizer 2000 (Malvern Instruments, Malvern, Worcestershire, UK) light scattering instrument. The Mastersizer 2000 uses an integrated optical system to cover the range from 0.02 to 2000 μm. The mixtures to be analysed is added to the test chamber filled with isopropanol until an obscuration of approximately 8-15% is reached. No ultrasound is applied in order not to alter the particle size distributions. The raw data is processed with the instrument software using the refractive index of the non nano-sized filler and applying the Mie correction together with the Fraunhofer approximation, frequently used techniques known to the expert.

### Particle Size Distribution (Nano-Sized Particles)

The measurement of the size of nano-particles is preferably based on a TEM (transmission electron microscopy) method, whereby a population is analysed to obtain an average particle diameter. A preferred method for measuring the particle diameter can be described as follows:

Samples approximately 80 nm thick are placed on 200 mesh copper grids with carbon stabilized formvar substrates (SPI Supplies—a division of Structure Probe, Inc., West Chester, PA). A transmission electron micrograph (TEM) is taken, using JEOL 200CX (JEOL, Ltd. of Akishima, Japan and sold by JEOL USA, Inc.) at 200 KV. A population size of about 50-100 particles can be measured and an average diameter is determined.

### Flexural Strength (FS)

If desired, the measurement of the flexural strength can be carried out according to ISO 4049 (2019) using a universal testing machine (Zwick Z 010, crosshead speed 2 mm/min) and test specimen having the size 2*2*25 mm. The flexural strength is typically given in MPa.

### Elongation at Break (EaB)

If desired, elongation at break of a material can be determined according to DIN EN ISO 527-1:2012-06. The elongation is given in % of the original length. Elongation data can be evaluated on a Zwick Z010 Universal testing machine by tearing at least three I-shaped specimens of the following dimensions: central unit: 10 mm×2 mm×2 mm; overall length: 25 mm; wider part width: 5 mm; radius of rounded edges: R=10 mm on the central unit; 25 mm on the wider part.

### Materials

TABLE 1

| Component | Abb. | Description | Source |
|---|---|---|---|
| Ionol ™ (2,6-di-tert•butyl-4- | STAB | stabilizer | BASF |
| methylphenol) | | | |
| Tinuvin ™ 326 | TV | UV light absorber | BASF |
| Lucirin ™ TPO | TPO | Photo-initiator | BASF |
| Camphor quinone | CQ | Photo-initiator | PCM Products |
| 1-Phenyl-1,2-propandion | PPD | Photo-initiator | Sigma |
| Benzil | BENZ | Photo-initiator | Sigma |
| Omnirad ™ 784 | 784 | Photo-initiator | Sigma |
| Ethyl 4-(dimethyl- | EDMAB | Co-initiator | Sarex Overseas |
| amino)benzoate | | | |
| Urethane methacrylate | DESMA | Polymerizable | cf. synthesis of compound (A1) of |
| | | monomer with | U.S. 2011/0053116 A1 (Hecht et al.) |
| | | urethane moieties | |
| D-Zethacrylate (ethoxylated | DZA | Polymerizable | |
| bisphenol A dimethacrylate) | | monomer without | |
| | | urethane moieties | |
| HDK ™ H-2000 | HDKH | Fumed silica; surface- | Wacker |
| | | treated | |
| Zr/Si nanocluster | Cluster | Filler; surface-treated | Zr/Si Nanocluster filler was |
| | | | produced as described in U.S. |
| | | | 6,730,156 B1, column 25, |
| | | | Preparatory Example A. The |
| | | | obtained filler particles were surface |
| | | | treated according to a process as |
| | | | described in Preparatory Example B |
| | | | of U.S. 6,730,156 B1. |

The light absorption properties of the photo-initiators are given in Table 2.

TABLE 2

| | Absorption in UV | Absorption in visible | |
|---|---|---|---|
| | light region (A_{UV}) | light region (A_{VIS}) | |
| Photo-Initiator | [390 nm] | [450 nm] | Ratio (A_{UV}/A_{VIS}) |
| TPO | 1.08 | 0 | n.a. |
| CQ | 0.11 | 1.08 | 0.1:1 |
| PPD | 1.25 | 0.58 | 2.2:1 |
| Benzil | 1.78 | 0.14 | 12.7:1 |
| Omnirad 784 | 1.3 | 1.09 | 1.2:1 |

TABLE 3

| Composition | RC1 (CE1) | RC2 (CE2) | RC3 (IE1) |
|---|---|---|---|
| STAB | 0.03 | 0.03 | 0.03 |
| TV | 0.10 | 0.10 | 0.10 |
| TPO | 0.80 | 0.80 | 0 |
| CQ | 0 | 0.20 | 0 |
| PPD | 0 | 0 | 0.80 |
| EDMAB | 0 | 1.12 | 1.26 |
| DESMA | 13.21 | 13.21 | 13.21 |
| DZA | 52.86 | 52.86 | 52.86 |
| HDKH | 2.50 | 2.50 | 2.50 |
| Cluster | 30.50 | 30.50 | 30.50 |
| Sum | 100.00 | 101.32 | 101.26 |
| Viscosity [1 s⁻¹] | 14.0 Pa*s | 10.4 Pa*s | 12.0 Pa*s |

values are given in party by weight;
CE: comparative example;
IE: inventive example

### General Process for Producing the Curable Composition

The respective components are mixed under save-light conditions using a speed mixer and followed by a roll mill step. In addition, the mixture is evacuated in a lab kneader.

### General Process of Producing 3D-Printed Articles

### Additive Manufacturing Process:

The composition is poured into the working tray of a commercially available DLP printer (Rapidshape, Heimsheim, Germany). The pre-processing data (STL-file; shape of 3-dimensional cuboid object; 25 mm*2 mm*2 mm) is loaded into the printer. The following printing conditions can be applied: curing light wavelength: 360-420 nm light; curing light intensity: 5-100 W/m²; exposure time: 1-11 sec; layer thickness: 25 μm.

### 3-Dimensional Article

The 3-dimensional article can be produced as follows: The composition is placed in a vat of an additive-manufacturing device. By using the parameters described above in the additive manufacturing process a 3-dimensional article is produced layer-by-layer. The 3-dimensional article may have the shape of a test sample or an orthodontic article, as desired. The 3-dimensional article was removed from the vat of the additive-manufacturing device.

### Cleaning Process:

The cleaning of the 3-dimensional article from excess material can be performed as described in WO 2019/023120 A1 (3M) using the parameters described in the text above.

### Post-Curing

An Elipar™ Deep-Cure S dental curing light (3M Oral Care) is used for conducting the post-curing step. This dental curing light provides light having a wavelength in the range of 430 to 480 nm. The following conditions were applied: 1470 mW/cm² for 60 s.

### Testing (General Process)

Using the formulations given in Table 3 specimens for flexural strength (FS) testing (2×2×25 mm) were prepared via the additive-manufacturing process described above, followed by a post-curing step. The results are given in Table 4.

TABLE 4

| | CE1 | CE2 | IE1 |
|---|---|---|---|
| FS after 3D printing [MPa] | 42.8 ± 2.0 | 32.7 ± 3.8 | not measurable; cf. FIG. 3 |
| FS after 3D printing & post-curing | 43.6 ± 2.1 | 73.0 ± 1.8 | 79.1 ± 4.4 |

The results in Table 4 show that TPO (CE1) can be used to obtain 3D printed specimens with a flexural strength of about 40 MPa.

An additional post-cure with visible light does not result in an increase of flexural strength.

Using a combination of TPO and camphor quinone (CE2) results in a flexural strength of about 30 MPa after 3D-printing and an increase to above 70 MPa after an additional treatment with visible light.

In contrast to that the use of PPD (IE1) results in a very flexible specimen after 3D-printing that cannot be destroyed by the testing machine (FIG. 3) and as such flexural strength cannot be determined.

Conducting an additional light-cure step increased the flexural strength to values of nearly 80 MPa.

This clearly shows that PPD can be used for 3D-printing of polymerizable composition into a highly flexible pre-cure state, which allows the manipulation and handling of the obtained article like it is possible with stainless steel crowns.

Further, a 3D-printed article in its pre-cured state has enough (meth)acrylate bonds available for achieving good adhesion with a bonding agent in case of a 3D-printed attachment, if desired.

The final cure (post-cure) can be realized with visible light in the mouth of a patient to ensure strength and durability.

1. A curable composition for use in a process of treating a dental situation in the mouth of a patient,
the curable composition comprising
radiation-curable component(s),
only photo-initiator(s) exhibiting an absorption in the UV light region and an absorption in the visible light region, the absorption at 390 nm being stronger than the absorption at 450 nm,
optionally filler(s),
optionally additive(s),
the process comprising the steps of
additive manufacturing a dental or orthodontic article layer-by-layer using radiation with a wavelength in the UV light region,
attaching the dental or orthodontic article to a surface of hard dental tissue or a dental material,
applying radiation with a wavelength in the visible light region to the dental or orthodontic article,
the UV light region being defined as light having a wavelength in the range of 350 to 410 nm,
the visible light region being defined as light having a wavelength in the range of 440 to 500 nm.

2. The curable composition for use according to claim 1, the photo-initiator(s)
comprising a di-ketone moiety, and/or
being non fluorescent.

3. The curable composition for use in particular according to claim 1, the photo-initiator(s) being selected from components comprising a phenyl-1,2-propanedione moiety, components comprising a benzil moiety, bis (Cyclopentadienyl) bis [2,6-difluoro-3-(1-pyrryl)phenyl titanium], monoacyl or diacylgermanium moiety and mixtures thereof.

4. The curable composition for use according to claim 1, the curable composition comprising only one photo-initiator, optionally in combination with an activator.

5. The curable composition for use according to claim 1 being characterized by a viscosity of <50 Pa*s at 23° C. and a shear rate of 1 s⁻¹.

6. The curable composition for use according to claim 1, the radiation-curable components being selected from (meth)acrylate components, urethane (meth)acrylate components and mixtures thereof.

7. The curable composition for use according to claim 1, the filler(s) comprising nano-sized filler particles.

8. The curable composition for use according to claim 1, the curable composition comprising the components in the following amounts:
radiation-curable component(s): 20 to 95 wt. %,
photo-initiator(s) optionally in combination with an activator: 0.01 to 5 wt. %,
filler(s): 0 to 70 wt. %,
additive(s): 0 to 10 wt. %.

9. The curable composition for use according to claim 1, the curable composition comprising the components in the following amounts:
methacrylate not comprising a urethane moiety: 30 to 65 wt. %,
urethane (meth)acrylates: 10 to 20 wt. %,
photo-initiator(s) optionally in combination with an activator: 0.02 to 4 wt. %,
filler(s): 15 to 50 wt. %,
additive(s): 0.1 to 5 wt. %,
wt. % with respect to the curable composition.

10. The curable composition for use according to claim 1, the curable composition comprising the components in the following amounts:
methacrylate not comprising a urethane moiety: 30 to 65 wt. %,
urethane (meth)acrylates: 10 to 20 wt. %,
photo-initiator(s) optionally in combination with an activator: 0.02 to 4 wt. %,
filler(s) comprising nano-sized filler particles with an average particle size of 40 nm or below: 15 to 50 wt. %,
additive(s): 0.1 to 5 wt. %,
wt. % with respect to the curable composition.

11. The curable composition for use according to claim 1,
wherein the dental or orthodontic article has a surface section for attaching it to the surface of a hard dental tissue or a dental material,
the process comprising the additional steps of
optionally post-processing the pre-cured dental or orthodontic article; and
applying a dental adhesive or dental cement to the surface section of the dental or orthodontic article intended to be attached to hard dental tissue or a dental material.

12. The curable composition for use according to claim 1, the process not comprising the additional steps of
roughening the surface section of the dental or orthodontic article intended to be attached to hard dental tissue or the dental material; and/or
applying a further radiation-curing step to the dental or orthodontic article before the dental or orthodontic article is attached to the hard dental tissue or the dental material.

13. A kit of parts comprising the curable composition for use according to claim 1, a dental adhesive or dental cement, optionally a dental positioning tray, and optionally an instruction of use.

14. A pre-cured composition obtainable by processing the curable composition for use according to claim 1 in an additive-manufacturing process, the pre-cured composition having the shape of a dental or orthodontic article.

15. The pre-cured composition according to the preceding claim being characterized by the following features alone or in combination:
being rubber-elastic;
having an elongation at break in the range of 10 to 200%, if determined according to DIN EN ISO 527-1:2012-06.
